(19) United States
(12) Patent Application Publication
(10) Pub. No.: US 2018/0042819 A1
(43) Pub. Date: Feb. 15, 2018

(54) Systems and Methods for Self-Detection Positioning of Nasogastric Tubes, Feeding Tubes, or Other Tubes

(71) Applicants: Brigham and Women's Hospital, Inc., Boston, MA (US); The General Hospital Corporation, Inc., Boston, MA (US)

(72) Inventors: Marc David Succi, Boston, MA (US); Steven Keating, Boston, MA (US); Allen Chen, Cambridge, MA (US); Christopher Trainor, Boxborough, MA (US); Brian Richard Daniels, Boston, MA (US); Leo Tsai, Arlington, MA (US); Jeffrey Ishizuka, Jamaica Plain, MA (US); Cheryl Cui, Boston, MA (US); Ali Tavakkoli, Boston, MA (US)

(21) Appl. No.: 15/557,804

(22) PCT Filed: Mar. 8, 2016
(86) PCT No.: PCT/US16/21358

§ 371 Date: Sep. 12, 2017

Related U.S. Application Data
(60) Provisional application No. 62/133,136, filed on Mar. 13, 2015.

(51) Int. Cl. A61J 15/00

(52) U.S. Cl. CPC A61J 15/0084; A61J 15/0026; A61B 5/14539

(57) ABSTRACT

A systems and methods for determining a position of a medical tube within a patient is provided. In one example, systems and methods are provided for a self- detection positioning system that is capable of determining a location of a distal end of a medical tube placed within a patient, and notifying a physician of the location of the distal end of the medical tube.

### CROSS-REFERENCES TO RELATED APPLICATIONS

The present application is based on, claims priority to, and incorporates herein by reference in its entirety, U.S. Provisional Patent Application No. 62/133,136, filed Mar. 13, 2015, and entitled “Systems and Methods For Self-Detection Positioning of Nasogastric Tubes, Feeding Tubes, Or Other Tubes.”

### STATEMENT REGARDING FEDERALLY SPONSORED RESEARCH

Not Applicable.

### BACKGROUND

The disclosure relates generally to medical devices used in conjunction with nasogastric, feeding, or other tubes and, more specifically, to a system and methods for self-detection positioning of such tubes.

Nasogastric (NG) tubes are a small plastic tube that can be inserted through the nares and ideally terminating in the stomach. NG tubes can be used to help decompress gas, to suction contents, to deliver medications, and for other therapeutic purposes. Similarly, feeding tubes are a small plastic tube, typically with a smaller diameter compared to NG tubes, inserted through the nares and ideally located in a post-pyloric position. Feeding tubes aim to deliver nutrition to patients that are not otherwise able to consume food through traditional means.

For both NG and feeding tubes, it is necessary to confirm that the tube is placed in the gastrointestinal tract (i.e., esophagus, stomach, intestines) as opposed to the pulmonary tract (i.e. trachea, bronchi). Aberrant, unintended placement in the lungs can cause considerable morbidity and possibly death. Unfortunately, incorrect placement of these tubes is a common and costly occurrence to both the patient and hospital system.

### BRIEF SUMMARY

The present disclosure provides a systems and methods for detecting the position of a nasogastric (NG) tube, a feeding tube, or other tube within a patient. In particular, systems and methods are provided for a self-detection positioning system that is capable of acquiring data that distinguishes positioning of a medical tube in the lungs or the gastrointestinal tract of a patient. The system is also capable of determining a specific section of the gastrointestinal tract (for example, the stomach or the duodenum) in which the medical tube is placed. Upon detecting the medical tube is properly positioned in the patient, a notification may be communicated to a trained individual indicating the medical tube has been properly placed.

In one aspect, the present disclosure provides a self-detection positioning system for a medical tube. The self-detection positioning system includes a casing configured to receive a proximal end of the medical tube and thereby place a distal end of the medical tube in communication with one or more sensors mounted within the casing. The self-detection positioning system further includes a controller mounted within the casing and in communication with the one or more sensors. The controller is configured to determine a location of the distal end of the medical tube within a patient based on data received from the one or more sensors.

In another aspect, the present disclosure provides a method for determining a position of a medical tube within a patient. The method includes inserting a distal end of the medical tube into the patient and placing the proximal end of the medical tube in communication with one or more sensors by inserting the proximal end of the medical tube into an adapter. The adapter is configured to receive the proximal end of the medical tube. The method further includes determining a position of the distal end of the medical tube within the patient based on data from the one or more sensors.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects and advantages other than those set forth above will become apparent when consideration is given to the following detailed description thereof. Such detailed description makes reference to the following drawings.

FIG. 1 is a schematic illustration of a self-detection positioning system in accordance with the present disclosure.

FIG. 2 shows a top view of the self-detection positioning system of FIG. 1.

FIG. 3 is a schematic illustration setting forth the steps for operating the self-detection positioning system of FIG. 1.

FIG. 4 illustrates one example of pressure data acquired by the self-detection positioning system of FIG. 1.

### DETAILED DESCRIPTION

Currently, confirmation of proper placement of a nasogastric (NG) tube, a feeding tube, or other tubes can be obtained using a stethoscope-mediated method, pH testing of fluid, or radiography. The stethoscope-mediated and pH testing methods are both cumbersome and often inaccurate. Radiographs are extremely sensitive and currently the gold-standard technique for tube placement confirmation. However, each radiograph requires a technologist to first obtain the images then a radiologist to interpret it, and this process is repeated every time a tube is placed, exchanged, or suspected to be malpositioned or malfunctioning; the financial cost of this technique is thus enormous for hospitals given the ubiquity of NG, feeding, and other medical tubes. Furthermore, every radiograph subjects the patient to an additional dose of ionizing radiation. Lastly, radiographic studies take time to acquire and interpret, during which a tube cannot be used, resulting in delay of clinical care. Given that misplacement of NG tubes within the bronchial tree has the potential to decrease pulmonary function and even cause respiratory compromise in tenuous patients, the time delay necessitated by obtaining radiographs could potentially negatively impact patient care.

Due to the current difficulties in cost consciously detecting proper placement of a nasogastric (NG) tube, a feeding tube, or other tube within the gastrointestinal tract of a patient, it would be desirable to have a low-cost, reusable, bedside, and self-detection positioning system that is capable of integration with current nasogastric (NG) tube, a feeding tube, or other tube. This would enable not only a physician, but also a nurse or other healthcare professional, to rapidly and reliably detect the position of a nasogastric (NG) tube, a feeding tube, or other tube at the bedside of a patient, obviating the need for the added expense, radiation dose, and delays associated with radiographs. Not only does the present disclosure provide such a system and method, but it does not require a proprietary tubing to operate and, thus, provides a cost-conscious solution to an enduring problem.

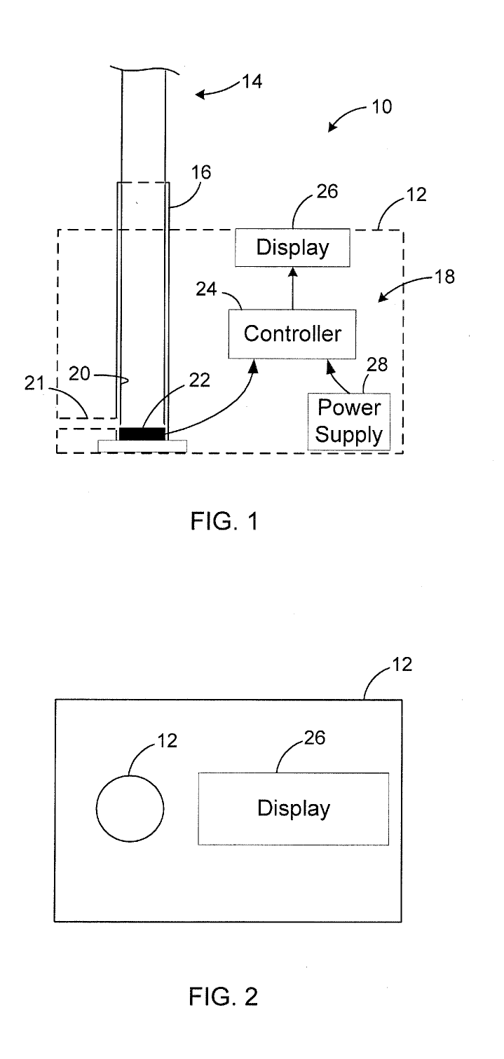

FIG. 1 shows a one non-limiting example of a self-detection positioning system 10 in accordance with the present disclosure. The self-detection system 10 includes a casing 12 which connects to a medical tube 14. The medical tube 14 may be any tube configured for insertion into a human body and including a lumen. For example, the medical tube may be a orogastric tube, a endotracheal tube, a chest tube, a urinary catheter, a nasogastric (NG) tube, or a feeding tube, to name a few. The casing 12 includes a removable adapter 16 received by the casing 12 and an electrical control system 18 mounted within the casing 12. The removable adapter 16 is configured to receive a proximal end 20 of the medical tube 14 and place one or more sensors 22 mounted within the removable adapter 16 in communication with a distal end (not shown) of the medical tube 14. An exhaust port 21 extending from the removable adapter 16 to the casing 12 provides a vent to atmospheric conditions enabling fluid to flow through the medical tube 14 and past the one or more sensors 22. As shown in the non-limiting example of FIG. 1, the removable adapter 16 protrudes from the casing 12. In another non-limiting example, the removable adapter 16 may be mounted flush with the casing 12.

The medical tube 14 can be a commercial nasogastric tube, a commercial feeding tube, or any other intubation tube known in the art. The proximal end 20 can be received by the removable adapter 14, as described above, while the distal end is typically inserted through the nares of a patient. Therefore, the one or more sensors 22 can acquire data from a location within the patient where the distal end of the medical tube 14 is positioned. In one non-limiting example, the one or more sensors 22 can include a pressure sensor, a temperature sensor, a pH sensor, and a humidity sensor. In other non-limiting configurations, the one or more sensors 22 may include more or less sensors configured to measure any physical, chemical, and/or electrical property, as desired.

The electrical control system 18 includes a controller 24 in communication with the one or more sensors 22 and display 26 in communication with the controller 24. The controller 24 can be reprogrammable to enable firmware updates to be installed on the controller 24. Additionally or alternatively, the controller 24 can be configured to communicate with a remote processor to receive firmware updates, process data received from the one or more sensors 22, and/or store data received from the one or more sensors 22. The controller 24 is powered by a power supply 28 and can be configured to relay the power, if necessary, to the one or more sensors 22 and/or the display 26. The power supply 28 can be a rechargeable battery, an AC to DC converter configured to receive wall power, or any other power supplying means known in the art. As shown in FIG. 1 and FIG. 2, the display 26 may be mounted adjacent to the removable adapter 16 on the casing 12. In other non-limiting configurations, the display 26 may be mounted anywhere on the casing 12, or the display 26 may be mounted remotely from the casing 12.

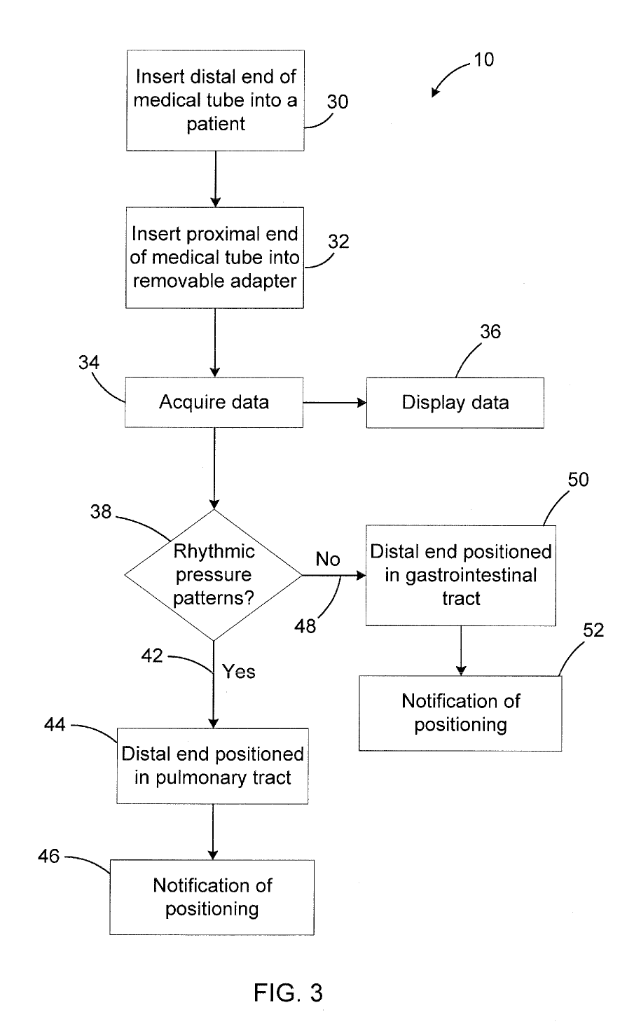

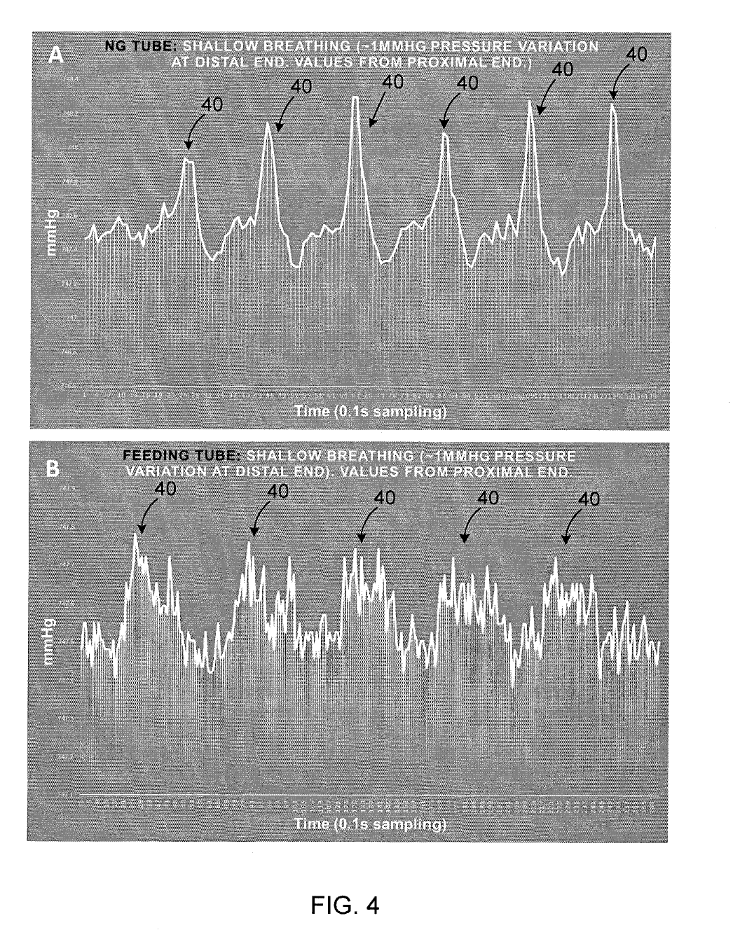

One non-limiting example of the operation of the self-detection positioning system 10 will be described with reference to FIGS. 1-4. In operation, the distal end of the medical tube 14 is inserted at step 30 into a patient, typically through the nares of the patient, by a trained individual. The trained individual may be a physician, nurse, MA, PA, or any other healthcare professional. Once the trained individual believes the distal end of the medical tube 14 is properly placed within the patient, the proximal end 20 of the medical tube 14 is inserted at step 32 into the removable adapter 16 thereby placing the distal end of the medical tube 14 in communication with the one or more sensors 22. The controller 24 can then acquire data at step 34 from the one or more sensors 22. In one non-limiting example, the controller 24 can acquire pressure data, temperature data, pH data, and humidity data from the one or more sensors 22. The controller 24 can instruct the display 26 to display at step 36 the data acquired from the one or more sensors 22 for viewing by the trained individual. The trained individual can review the data and determine if the distal end of the medical tube 14 is properly placed within the patient. Additionally or alternatively, the controller 24 can be configured to detect at step 38 physiological rhythmic patterns 40, as illustrated in two non-limiting examples of pressure data shown in FIG. 4. Upon detection of physiological rhythmic pressure patterns 40 indicated by arrow 42, the controller 24 can determine at step 44 that the distal end of the medical tube 14 is located in a pulmonary tract of the patient and subsequently notify at step 46 the trained individual using the display 26 of this location. If rhythmic pressure patterns 40 are not detected indicated by arrow 48, the controller 24 can determine at step 50 that the distal end of the medical tube 14 is located in a gastrointestinal tract of the patient and subsequently notify the trained individual using the display 26 of this location at step 52. Alternatively or additionally, the controller 24 can be configured to detect at step 38 physiological patterns from any of the one or more sensors 22. In some instances, the medical tube 14 can be placed into the patient by a first trained individual and placement of the medical tube 14 can be confirmed by a second trained individual. In this instance, the second trained individual will monitor the display 26 and determine the location of the distal end of the medical tube 14, once the first trained individual believes the distal end of the medical tube 14 is properly placed within the patient.

In other non-limiting examples, the controller 24 can be configured to detect humidity, temperature, and/or pH data in combination with, or separately from, the pressure data to determine the location of the distal end of the medical tube 14. For example, the controller 24 can detect no rhythmic pressure patterns 40 and elevated humidity and determine that the distal end of the medical tube 14 is located in the gastrointestinal tract of the patient. Alternatively or additionally, the controller 24 can be configured to determine a specific section of the gastrointestinal tract in which the distal end of the medical tube 14 is located, based on data from the one or more sensors 22. For example, the controller 22 may be configured to detect whether the distal end of the medical tube is located within a duodenum or a stomach of the gastrointestinal tract of the patient. Furthermore, the controller 24 can be configured to detect any physical, chemical, and/or electrical data from the one or more sensors 22 to determine the location of the distal end of the medical tube 14.

The notifications at steps 46 and 52 sent to the display 26 notifying the trained individual of the positioning of the distal end of the medical tube 14 may be in the form of a binary output, where one output signifies positioning in the pulmonary tract and the other output signifies positioning in the gastrointestinal tract. Alternatively or additionally, the display 26 may articulate the positioning of the distal end of the medical tube 14 to the physician.

Exemplary advantages of the above-described self-detection positioning system 10 or other medical systems designed or created using the above-described techniques or properties, will be described with reference to FIGS. 1-4. By no means is the following an exhaustive list of the numerous advantages provided by the invention, as will be understood by one of skill in the art.

The self-detection positioning system 10 provides quick and accurate determination of a position of a distal end of a medical tube 14 placed in a patient. The accuracy of the self-detection positioning system 10 may negate the need for a radiologist to confirm the placement of the medical tube 14, and enable a less expensive medical professional, for example, a nurse, MA, PA, or other health care professional to confirm the placement of the medical tube 14.

The components 16, 22, 24, 26, 28 mounted within or on the casing 12 can be easily configured to fit in a compact design enabling a physician to use the self-detection positioning system 10 in a point-of-care manner, or at the patient's bedside. Additionally, the removable adapter 16 received by the casing 12 is removable from the casing 12 enabling the self-detection positioning system 10 to be reusable and prevent cross-contamination between patients.

As described above, the self-detection positioning system 10 can use one or more sensors 22 to detect specific patterns in data to determine the position of the distal end of the medical tube 14. Since the data measured by the one or more sensors 22 from within the patient will most likely be at substantially atmospheric conditions, the one or more sensors 22 can be low-cost and easily adapted to fit in a compact design.

Furthermore, the controller 24 can be reprogrammed enabling the controller 24 to adaptively learn. This adaptive learning could allow the self-detection positioning system 10 to more precisely determine the position of the distal end of the medical tube 14. For example, the controller 24 could be configured to determine, based on the data from the one or more sensors 22, whether the distal end of the medical tube 14 was positioned in a post-plyoric position within a gastrointestinal tract of a patient.

Although the above description generally relates to applying the self-detection positioning system 10 when inserting a nasogastric and/or feeding tube in a patient, they are but two non-limiting applications of the self-detection positioning system 10. In other non-limiting applications, the self-detection positioning system 10 could be applied during any process of inserting a medical tube into a patient known in the art.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A self-detection positioning system for a medical tube, the self-detection positioning system comprising:
a casing configured to receive a proximal end of the medical tube and thereby place a distal end of the medical tube in communication with one or more sensors mounted within the casing; and
a controller mounted within the casing and in communication with the one or more sensors, wherein the controller is configured to determine a location of the distal end of the medical tube within a patient based on data received from the one or more sensors.

2. The self-detection positioning system of claim 1, wherein the one or more sensors includes at least one of a pressure sensor, a temperature sensor, a pH sensor and a humidity sensor.

3. The self-detection positioning system of claim 2, wherein the controller is further configured to detect physiological rhythmic patterns in the data.

4. The self-detection positioning system of claim 2, wherein the controller is further configured to determine that detecting physiological rhythmic pressure patterns indicates the distal end of the medical tube is located in a pulmonary tract of the patient.

5. The self-detection positioning system of claim 3, wherein the controller is further configured to determine that not detecting the physiological rhythmic pressure patterns indicates the distal end of the medical tube is located in a gastrointestinal tract of the patient.

6. The self-detection positioning system of claim 3, wherein the controller is further configured to determine that not detecting the physiological rhythmic pressure patterns and detecting elevated humidity indicates the distal end of the medical tube is located in a gastrointestinal tract of the patient.

7. The self-detection positioning system of claim 1 further comprising a display in communication with the controller.

8. The self-detection positioning system of claim 7, wherein the display is configured to display the location of the distal end of the medical tube determined by the controller.

9. The self-detection positioning system of claim 7, wherein the display is mounted on the casing.

10. The self-detection positioning system of claim 7, wherein the display is configured to display the data from the one or more sensors.

11. The self-detection positioning system of claim 1 further comprising a removable adapter received by the casing and configured to receive the proximal end of the medical tube.

12. The self-detection positioning system of claim 1, wherein the controller is further configured to determine a specific section of a gastrointestinal tract of the patient in which the distal end of the medical tube is located.

13. A method for determining a position of a medical tube within a patient, the method comprising:
inserting a distal end of the medical tube into the patient;
placing a proximal end of the medical tube in communication with one or more sensors by inserting the proximal end of the medical tube into an adapter, wherein the adapter is configured to receive the proximal end of the medical tube; and
determining a location of the distal end of the medical tube within the patient based on data from the one or more sensors.

14. The method of claim 13, wherein the data from the one or more sensors includes at least one of pressure data, temperature data, pH data, and humidity data.

15. The method of claim 14, wherein the location of the distal end of the medical tube is determined by the pressure data.

16. The method of claim 14, wherein the location of the distal end of the medical tube is determined by the pressure data and the humidity data.

17. The method of claim 14 further comprising detecting a physiological rhythmic pattern in the data and determining the location of the distal end of the medical tube is in a pulmonary tract of the patient.

18. The method of claim 14 further comprising detecting no physiological rhythmic pressure pattern in the data and determining the location of the distal end of the medical tube is in a gastrointestinal tract of the patient.

19. The method of claim 14 further comprising detecting no physiological rhythmic pressure pattern and elevated humidity in the data and determining the location of the distal end of the medical tube is in a gastrointestinal tract of the patient.

20. The method of claim 13 further comprising displaying the location of the distal end of the medical tube on a display.

21. The method of claim 13 further comprising displaying the data from the one or more sensors.

22. The method of claim 20, wherein the display is mounted on a casing associated with the one or more sensors.

23. The method of claim 13 further comprising indentifying a specific section of a gastrointestinal tract of the patient in which the distal end of the medical tube is located.
